(19) United States
(12) Patent Application Publication
(10) Pub. No.: US 2016/0338584 A1
(43) Pub. Date: Nov. 24, 2016

(54) APPARATUS AND METHOD FOR INDUCING POLARIZATION PERCEPTION IN AN OBSERVER

(71) Applicant: Vizeye Limited, West Midlands (GB)

(72) Inventors: Peter John Bryanston-Cross, Coventry (GB); Gary Philip Misson, Stratford-upon-Avon (GB); Brenda Timmerman, Amersfoort (NL)

(21) Appl. No.: 15/104,386

(22) PCT Filed: Dec. 19, 2014
(86) PCT No.: PCT/GB2014/053789

§ 371 Date: Jun. 14, 2016

(30) Foreign Application Priority Data
Dec. 20, 2013 (GB) 1322671.7

(51) Int. Cl. A61B 3/02; G02F 1/1333; G02F 1/01; A61B 3/00

(52) U.S. Cl. CPC A61B 3/02; A61B 3/0008; A61B 3/0041; G02F 1/1333; G02F 1/0136; A61B 2560/0431

(57) ABSTRACT

Apparatus for inducing polarization perception in a human or animal observer. The apparatus may comprise several light emitting diodes (LEDs) (4), an adjacent planar light scattering surface (6), a preferably blue colour filter (88), a sheet polarizer (10), and a liquid crystal cell (LCC) array (12). The absence of a secondary polarizer after the LCC layer means that the polarization state of the light exiting the LCC array (12) is preserved. Each cell of the LCC array (12) can therefore be controlled such that a polarization pattern, e.g. a checkerboard pattern, is produced. The polarization pattern may be temporally variable, e.g. alternating between a sequence of patterns.

### FIELD OF THE INVENTION

The present invention relates to an apparatus and method for inducing polarization perception in an observer. The apparatus comprises a polarization manipulation layer through which light passes.

### BACKGROUND OF THE INVENTION

A common cause of blindness in the Western world is age-related macular degeneration. Other diseases of the macular part of the retina of the eye, such as diabetic retinopathy, are also significant causes of blindness. There are treatments for some patients with these conditions and the best results are from early intervention before the disease has caused permanent damage. Early detection, and preferably self-detection, is therefore beneficial to the patient and to the health service in general as early treatment results in reduced disability.

Haidinger's phenomenon (HP), also known as Haidinger's brush or brushes, is a visual perception of light polarization. Haidinger's phenomenon may be perceived by humans as a faint yellow/blue hour-glass shape on viewing linearly polarized white light. HP is most readily perceived when observing blue linear polarized light (peak wavelength: 460 nm). Under these conditions, HP is seen as a dark hour-glass like image, as shown in FIG. 1. The image has its dark components perpendicular to the direction of polarization. The phenomenon is difficult to observe as it is very faint and rapidly fades because of local retinal adaptation effects (the Troxler phenomenon).

HP is induced at the polarization sensitive region of the macula of the retina and its polarization and spectral characteristics are due to the yellow macular xanthophyll pigments. The two most common of these in the macula are lutein and zeaxanthin, which are pleochroic (exhibiting different colours when viewed from different angles). Due to the molecular structure of these pigments a proportion of the molecules naturally align with the radially orientated fibres of the photoreceptors in the macular region of the retina (Henle fibre layer). Selective absorption of linearly polarized light is maximal for pigment molecules orientated orthogonal to the direction of polarization of incident light. For other angles the light transmission approximately obeys Malus' law, i.e. transmitted light intensity is proportional to the square of the cosine of the angle between the direction of linear polarization and the pleochroic molecular alignment.

Under appropriate conditions HP can be seen by most subjects having normal eyes and vision. However, HP is not seen by eyes that have any condition which disturbs the radially symmetric array of the Henle fibres. Common treatable macular diseases such as wet age related macular degeneration (wAMD) and diabetic maculopathy (DM) are known to negate HP. A wide range of less common macular conditions also affect/negate the perception of HP, including macular oedema and macular atrophy.

The consensus is that light polarization perception (PP) is a sensitive macular diagnostic test, but is not specific for any particular disorder. It has potential as a useful screening test for macular disease particularly in patients known to be in the early stages e.g. of AMD. HP is difficult to see and existing apparatus for detection of HP are not easy to use. Accordingly, there is a need for an apparatus which can be used to induce the perception of polarization, and which may be portable, simple in terms of including only a few mechanical components and therefore cheap, and also simple to use.

### SUMMARY OF THE INVENTION

A first aspect of the invention provides an apparatus for inducing polarization perception in an observer, the apparatus comprising:
a light transmitting part configured to transmit light of substantially uniform luminance and colour;
a polarization manipulation layer comprising a polarizing structure having one or more regions of uniform state of polarization and arranged such that light transmitted through the light transmitting part passes through the layer; and
means for causing discrete changes in the state of polarization of the polarizing structure.

This apparatus allows the polarization of light to be clearly and continuously perceived by an observer with healthy eyes. Thus this apparatus can be used as a means for testing eye health, for example to test macular function. The discrete changes in the state of polarization of the polarizing structure mean that the polarization sensitive regions of the eye of the observer are exposed to changes in light polarization. This counteracts the tendency of the eye to adjust so as to reduce the perception of polarization. Using light of substantially uniform luminance and colour ensures that the perceived effect is due only to polarization, since significant differences in luminance or colour could overwhelm the more subtle polarization perception phenomenon.

The light transmitting part may comprise a light emitting assembly configured to emit light of substantially uniform luminance and colour and the polarization manipulation layer may be arranged such that light emitted from the light emitting assembly passes through the layer. Integrating a light emitting assembly with the apparatus removes the need for an external light source. This allows the apparatus to be embodied in a single device. The properties of the light can also be more carefully controlled.

The light emitting assembly may comprise one or more light sources and a chromatic filter. Using a chromatic filter produces light of a very narrow waveband. The colour and temperature response of the light sources does not need to be as carefully controlled when the chromatic filter is present. Thus cheaper light sources may be used which reduces the overall cost of manufacturing the apparatus. The light emitting assembly may comprise a linear polarizer.

The polarizing structure may have a plurality of regions and each region may have a uniform state of polarization which differs from the state of polarization of each adjacent region. Providing a plurality of adjacent regions having different states of polarization results in at least one boundary between differently polarized areas being visible to an observer of the apparatus. As the observer's focus moves across the polarization manipulation layer, either deliberately or through involuntary saccadic eye movements, the polarization sensitive regions of the macula are exposed to changes in light polarization. This counteracts the tendency of the eye to adjust so as to reduce the perception of polarization.

The polarizing structure may define a polarization pattern and the means for causing discrete changes in the state of polarization of the polarizing structure may comprise means for producing a series of at least two different polarization patterns. Using polarization patterns or images may make the polarization of light easier to perceive. The pattern may be an easily recognizable and describable one (as opposed to the plain Haidinger's phenomenon, which may appear indistinct to many observers). Providing means for producing a series of at least two different polarization patterns allows thorough testing of an observer using a single apparatus. Not all observers have the same ability to perceive light polarization or the same distribution of polarization sensitive regions and so some patterns may be clearer than others to different observers. The allows the potential for diagnostic and other quantification. The means for causing discrete changes in the state of polarization of the polarizing structure may be configured to cause two or more different polarization patterns to be produced sequentially.

A frequency at which the different polarization patterns are produced may be in the range of 1-10 Hz. Such a frequency does not allow the eye time to adjust so as to diminish the observer's perception of polarization.

At least one of the series of polarization patterns may comprise an array of linearly polarized regions. The array may comprise an array of squares (i.e. a checkerboard). This array pattern results in multiple boundaries between areas of different state of polarization being in the field of view of an observer, enhancing the polarization perception effect.

At least one of the series of polarization patterns may comprise a symbol. Symbols are easily recognised and described by observers of most ages and abilities, which aids in testing and diagnosis. Subjects are also accustomed to identifying symbols during conventional vision tests.

The means for causing discrete changes in the state of polarization of the polarizing structure may comprise a controller for controlling changes in the state of polarization of the polarizing structure. The controller may for example be a microprocessor. This allows the apparatus to be implemented in modern electronic devices and to be pre-programmed and re-programmable.

The means for causing discrete changes in the state of polarization of the polarizing structure may comprise one or more user inputs. This allows a user or health care professional to control operation of the device, including indicating whether a polarization pattern can be perceived or not by the observer, controlling the time/frequency of changes in the polarization pattern presented and the luminance, powering the apparatus on/off, inputting data/results and causing data/results to be sent to an external device.

The apparatus may be a portable display device. This enables people to use the device for self testing of macula function/health. This is particularly advantageous for those people at risk of developing macula disorders and where early detection of a deterioration in macula function could improve the effectiveness of treatment.

The polarization manipulation layer may comprise an array of liquid crystal cells. Liquid crystal cells can be controlled individually and electronically and the technology is readily available allowing the device to be manufactured cheaply.

A second aspect of the invention provides an apparatus for inducing polarization perception in an observer, the apparatus comprising:
a light transmitting part configured to transmit light of substantially uniform luminance and colour; and
a polarization manipulation layer comprising a polarizing structure having a plurality of regions and arranged such that light transmitted through the light transmitting part passes through the layer;
wherein each region of the polarizing structure has a uniform state of polarization which differs from the state of each adjacent region.

This apparatus allows the polarization of light to be clearly and continuously perceived by an observer with healthy eyes. Thus this apparatus can be used as a means for testing eye health, for example to test macular function. Providing a plurality of adjacent regions having different states of polarization results in at least one boundary between differently polarized areas being visible to an observer of the apparatus. As the observer's focus moves across the pattern generated by the polarization manipulation layer, either deliberately or through involuntary saccadic eye movements, the polarization sensitive regions of the macula are exposed to changes in light polarization. This counteracts the tendency of the eye to adjust so as to reduce polarization perception.

Using light of substantially uniform luminance and colour isolates the observer's ability to perceive the polarization of the light, since significant differences in luminance or colour may overwhelm the more subtle polarization perception phenomenon.

The light transmitting part may comprise a light emitting assembly configured to emit light of substantially uniform luminance and colour and the polarization manipulation layer may be arranged such that light emitted from the light emitting assembly passes through the layer. Integrating a light emitting assembly with the apparatus removes the need for an external light source. This allows the apparatus to be embodied in a single device. The properties of the light can also be more carefully controlled.

Alternatively, the light transmitting part may comprise a colour filter configured to receive ambient light. Harnessing ambient light means that the apparatus requires no power supply. For example the polarization manipulation layer may be embossed or otherwise integrated into a material along with the colour filter. The apparatus may then be held up to an ambient light source by a user in order to observe the polarization pattern of this layer. All of the components of the apparatus may be flexible.

The plurality of regions of the polarizing structure may define a polarization pattern. Using polarization patterns or images may make the polarization of light easier to perceive. The pattern may be an easily recognizable and describable one (as opposed to the plain Haidinger's phenomenon, which may appear indistinct to many observers).

The polarization pattern may comprise an array of linearly polarized regions. The array may comprise an array of squares (i.e. a checkerboard). This array pattern results in multiple boundaries between areas of different states of polarization being in the field of view of an observer, enhancing the polarization perception effect. The polarization pattern may comprise one or more symbols. The symbol may be an alphanumeric symbol. Symbols are easily recognised and described by observers of most ages and abilities, which aids in testing and diagnosis. Subjects are also accustomed to identifying symbols during conventional vision tests. The polarization pattern may also comprise an image or discrete pattern more complex than those previously mentioned, for example a human face.

A third aspect of the invention provides a method of using a display device for inducing polarization perception in an observer, the method comprising:
providing a light transmitting part configured to transmit light of substantially uniform luminance and colour;
providing a polarization manipulation layer comprising a polarizing structure having a plurality of regions of uniform state of polarization and arranged such that light transmitted through the light transmitting part passes through the layer;
arranging the display device such that at least one boundary between adjacent regions is projected onto the polarization sensitive region of the macula of an observer.

This method allows the polarization of light to be clearly and continuously perceived by an observer with healthy eyes. Thus this method can be used as a means for testing eye health, for example to test macular function. Arranging the display device such that at least one boundary between adjacent regions is projected onto the polarization sensitive region of the macula of an observer means that a contrast in polarization states can be perceived by the observer. As the observer's focus moves across the pattern generated by the polarization manipulation layer, either deliberately or through involuntary saccadic eye movements, the polarization sensitive regions of the macula are exposed to changes in light polarization. This counteracts the tendency of the eye to adjust so as to reduce the perception of polarization.

Using light of substantially uniform luminance and colour isolates the observer's ability to perceive the polarization of the light, since significant differences in luminance or colour may overwhelm the more subtle polarization perception phenomenon.

The light transmitting part may comprise a light emitting assembly configured to emit light of substantially uniform luminance and colour and the polarization manipulation layer may be arranged such that light emitted from the light emitting assembly passes through the layer. Integrating a light emitting assembly with the apparatus removes the need for an external light source. This allows the apparatus to be embodied in a single device. The properties of the light can also be more carefully controlled.

The plurality of regions of the polarizing structure may define a polarization pattern. The method may further comprise arranging the display device such that the polarization pattern occupies a visual angle sufficient for the pattern to be projected onto the polarization sensitive part of the macula which is typically between 2 and 5 degrees of visual angle from the point of view of the observer.

A fourth aspect of the invention provides an apparatus for inducing polarization perception in an observer, the apparatus comprising:
a light transmitting part configured to transmit light of substantially uniform luminance and colour; and
a polarization manipulation layer comprising a polarizing structure having one or more regions and arranged such that light transmitted through the light transmitting part passes through the layer;
wherein each region of the polarizing structure has a uniform state of polarization which differs from the polarization stale of each adjacent region and wherein the apparatus is configured to vary the state of polarization of the polarizing structure wherein the variances are temporal and/or spatial.

Embodiments of the invention will now be described, by way of example only, with reference to the accompanying drawings.

### BRIEF DESCRIPTION OF THE FIGURES

FIG. 1 shows a simulation of Haidinger's phenomenon at three different polarization orientations, indicated by arrows;

FIG. 2 shows schematically an apparatus for inducing polarization perception in an observer according to embodiments of the invention;

FIG. 3 illustrates the underlying principles of twisted nematic liquid crystal cells and the effect on the polarization state of light passing through the cells;

FIG. 4a shows an input pattern which can be created using the apparatus of FIGS. 2 and 3 (note black and white represent different states of polarization, for example black may be horizontal linear polarization and white may be vertical linear polarization);

FIG. 4b shows diagrammatically the individual liquid crystal cells for a portion of the input pattern of FIG. 4a and indicates their respective polarization orientation;

FIGS. 5a and 5b show further input patterns which can be created using the apparatus of FIGS. 2 and 3 and also mathematical simulations of the expected appearance to a healthy observer of these input patterns;

FIG. 6 shows a number of different polarization patterns and simulations of their respective expected appearances; and

FIG. 7 illustrates a further embodiment in which the polarized images are projected onto a polarization preserving surface before being viewed by one or more observers.

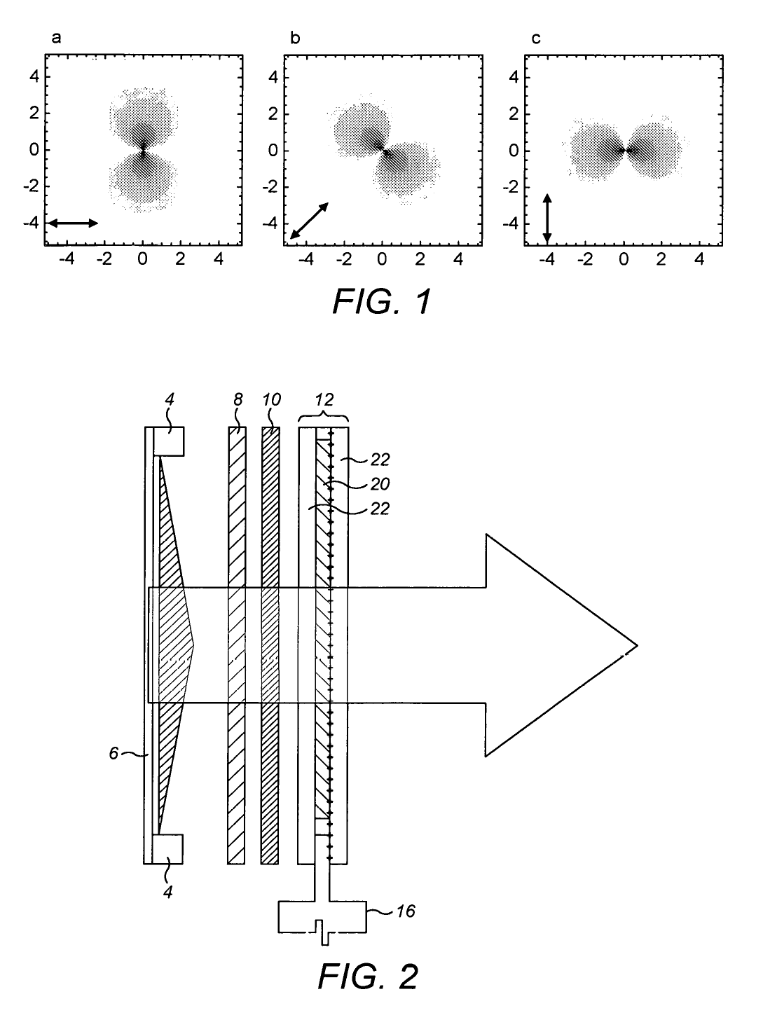

### DETAILED DESCRIPTION OF SOME EMBODIMENTS OF THE INVENTION

FIG. 2 shows a simplified schematic diagram of an apparatus 2 for inducing polarization perception in an observer according to some embodiments of the invention.

In order to perceive light polarization, a light source is required, and this may be provided by a number of different ways. In the embodiment that is illustrated in FIG. 2, the apparatus includes several light emitting diodes (LEDs) 4, which diffusely illuminate an adjacent planar light scattering surface 6. For example, the light scattering surface may be a mirrored surface or a light guide. In some embodiments, the LEDs 4 emit blue light at a peak wavelength of approximately 460 nm. This is the wavelength at which the polarization of light is best perceived. The light source may comprises one or more monochromatic sources.

The apparatus 2 also comprises a colour filter 8, for example a sheet filter, and a sheet polarizer 10. Light emitted from the light scattering surface 6 may pass first through the colour filter 8 and then through the polarizer 10. Alternatively, the order of these components may be switched. In some embodiments, the filter 8 is a blue filter, for example LEE filter 075 ‘evening blue’, manufactured by Lee Filters. The colour filter 8 ensures that light is produced of the appropriate wavelength for optimal perception of the polarization of light. The sheet polarizer 10 may linearly polarize light which passes through it. In some embodiments, the sheet polarizer 10 may be a dichroic polarizing filter which passes only a narrow band of wavelengths. This may remove the need for the colour filter 8 in these embodiments and also reduce the amount of light which is absorbed or reflected before reaching the observer. The LEDs 4, filters and polarizers may together be referred to as a light emitting assembly.

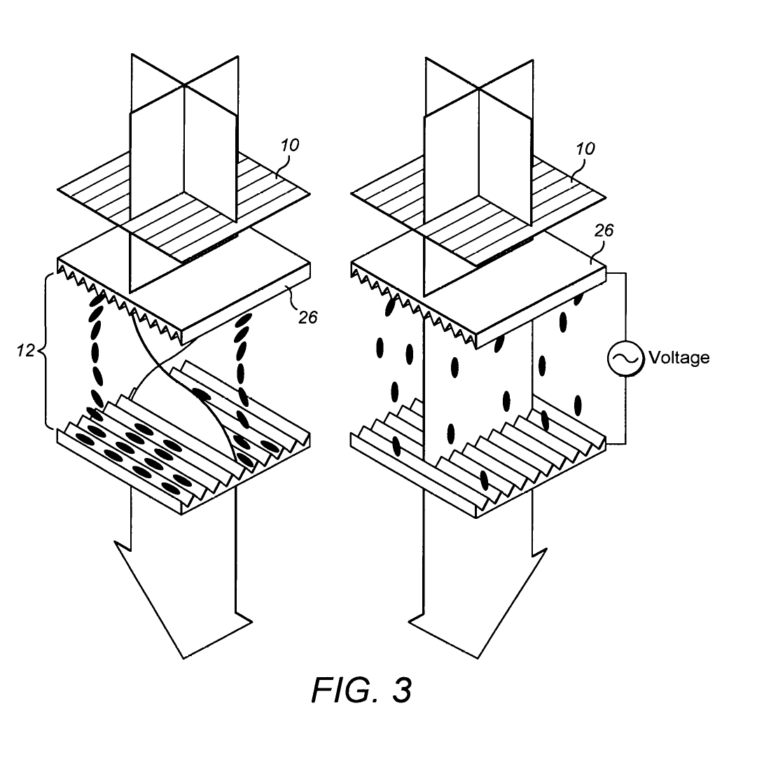

The apparatus 2 also comprises a liquid crystal cell (LCC) array 12. Referring to FIGS. 2 and 3, the LCC array 12 is made of conventional (e.g. twisted nematic) materials, and comprises a layer of liquid crystal material 20 divided into cells (pixels) and sandwiched between two flat and parallel glass sheets 22. The inner faces of the glass sheets 22 are coated with a thin, transparent, electrically conductive layer (e.g. indium tin oxide). A further layer, known as an alignment layer 26, is deposited on the conductive layer, and imposes an orientation on the adjacent liquid crystal molecules, as shown in FIG. 3. The LCC array 12 has a control circuit 16, allowing the orientation of the liquid crystal molecules to be controlled. A transparent glass or plastic screen (not shown) may cover the LCC array 12. The LCC array 12 may be based on a thin film transistor LCD design or any other appropriate LCD design. The LCC array 12 may also be referred to herein as a polarization manipulation layer or polarization controlling layer.

For example in one of the standard modes of operation, when no potential is applied to a LCC, the twisted configuration of the liquid crystal molecules causes a 90 degree change in the polarization orientation of the transmitted light. When a potential is applied to the LCC, the liquid crystal molecules are aligned with the resulting electric field and the light retains its original polarization orientation. Depending on LCC design, linear polarization orientations in between these two states can be achieved by varying the applied electrical potential. In other LCC designs, elliptically or circularly polarized light can also be produced by correctly modulating the applied potential. In general, when “inactive” (no potential applied), the polarization state of light exiting the LCC is determined by the orientation of the sheet polarizer 10 and the particular characteristics of the LCC. When “activated” (potential applied), the polarization state of light exiting the LCC is dependent on the applied voltage, the waveform of the voltage and the characteristics of the LCC. In addition or as an alternative to the light scattering surface 6, the apparatus 2 may have optics to produce a collimated light beam. The apparatus may also include a monochromator to produce light of substantially uniform colour. In some embodiments the monochromator may replace the colour filter 8. The monochromator may additionally or alternatively replace the sheet polarizer 10, provided that the monchromator also produces polarized light. In some embodiments, the sheet polarizer 10 may be omitted and the polarization state of the light may be controlled only by the liquid crystal cell layer 12. In general, the apparatus 2 is most effective at inducing the perception of polarization in an observer when producing light of substantially uniform luminance and colour. If the variance in the luminance and colour between two regions of the LCC array 12 having different states of polarization is too great, then an observer will not be able to distinguish between an effect due to luminance and an effect due to polarization.

The light source could, in some alternative embodiments, be ambient light (e.g. day light) when combined with a suitable optical system to produce a homogeneously illuminated display area. Thus the LEDs 4 may be replaced with a more general light transmitting part configured to harness and direct ambient light (natural or artificial) to the LCC array 12. For example, a transparent light diffuser and colour filter may be used in combination with an LCC layer or other type of polarization manipulating layer. A sheet polarizer may also be used to enhance the effect.

The inventors have developed several different embodiments for using the apparatus 2 shown schematically in FIG. 2. Each of the cells in the LCC array 12 is individually addressable, in much the same way as the pixels of a LCD screen. However each of the cells in the array produces light of substantially the same brightness and colour, such that a display area is produced which appears homogeneous. The absence of a secondary polarizer after the LCC layer means that the polarization state of the light exiting the LCC array 12 is preserved. Each cell of the LCC array 12 can therefore be controlled such that a “polarized image” (also referred to herein as a polarization pattern and a polarized background image/pattern) is produced.

Depending on the intended method of use and cost/complexity considerations, the apparatus 2 can be used in at least three modes:
Static mode, involving spatial modulation of the polarization pattern
Active mode, involving temporal modulation of the polarization pattern
A combination of the static and active modes of operation.

Firstly, the static mode of operation will be described. In this mode a single polarized image/pattern is produce by the apparatus 2. This polarization pattern comprises a plurality of different regions which are distinguished from adjacent regions by having a different state of polarization. A healthy observer viewing at the correct distance and angle is then able to perceive the polarization pattern. The terms “state of polarization”, “polarization state” and “polarization orientation” are used interchangeable herein. The state of polarization of the regions of the polarizing structure and of the light exiting these regions may be linear, elliptical or circular. Similarly the polarization orientation may be linear, elliptical or circular. This applies to all embodiments described herein.

Typical values for the dimension of the polarization sensitive macular pigment distribution region are of the order of 1.5 mm diameter around the fovea of diameter 0.6 mm. Thus, for a polarization pattern image to be fully visualised by a normal macula the projected polarization pattern should be smaller than this pigment distribution area. This corresponds to a suitable perceived image angle for distant objects to subtend up to 5 degrees (which will have a projected image diameter of approximately 1.4 mm on the retina), similar as typically found for Haidinger's phenomenon.

For a contrast between areas of different state of polarization to be perceived by an observer, at least one boundary between areas of different state of polarization must fall within this region, i.e. there must be at least one discreet polarization step incident on the polarization sensitive region of the macula (although polarized images may extend beyond the polarization sensitive region of the macula).

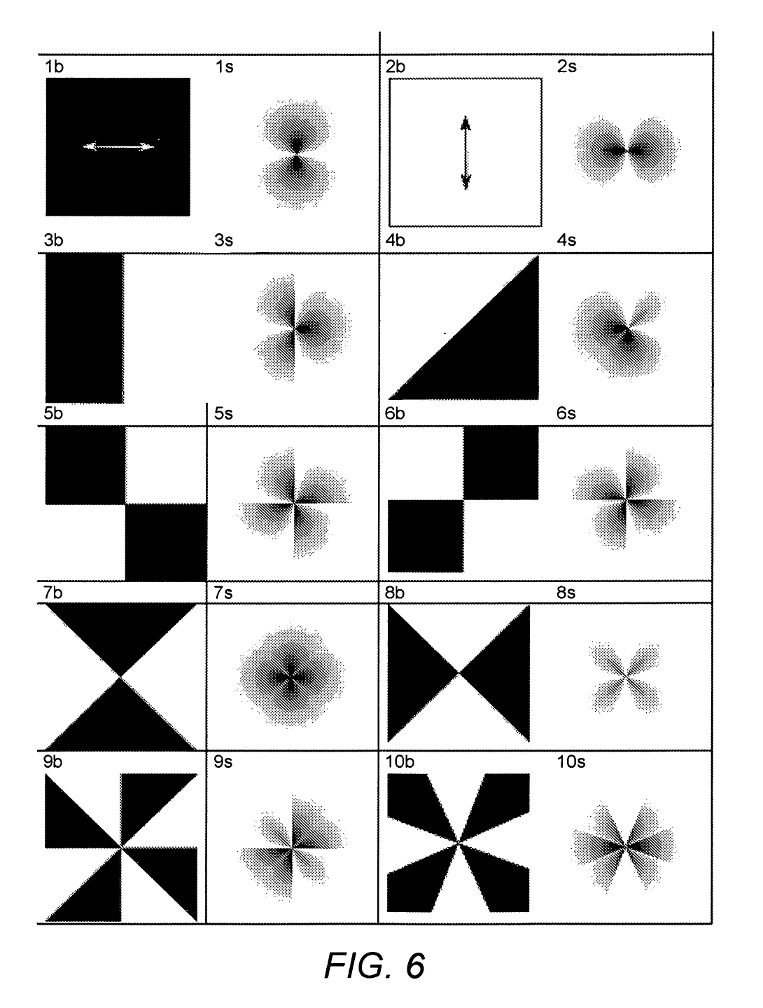

With reference to FIG. 6, which shows a number of different polarization patterns and simulations of their respective expected appearances, some embodiments will now be described. The simplest continuously perceivable embodiment of the static mode of operation is two adjacent areas having orthogonal polarization, for example horizontal and vertical respectively (FIG. 6 (1b) and (2b)). In this case, an observer would see a vertical Haidinger's brush when looking at one half of the display area (FIG. 6 (1s)) and a horizontal Haidinger's brush when looking at the other half of the display area (FIG. 6 (2s)). These images will fade rapidly because of the Troxler phenomenon as above unless gaze is alternated between areas. When observing the boundary of the areas (FIG. 6 (3b)) the observer will see a modified HP image comprising bisected components of the vertical and horizontal HP of each half (FIG. 6 (3s); the boundary between the adjacent areas being accentuated because of the abrupt change in contrast at the bisection of the modified HP image. The image is perceived as continuous because the Troxler phenomenon is overcome by saccadic eye movements across the boundary. A device capable of presenting such a polarization pattern can be created cheaply and can be effective as a general test of macula function.

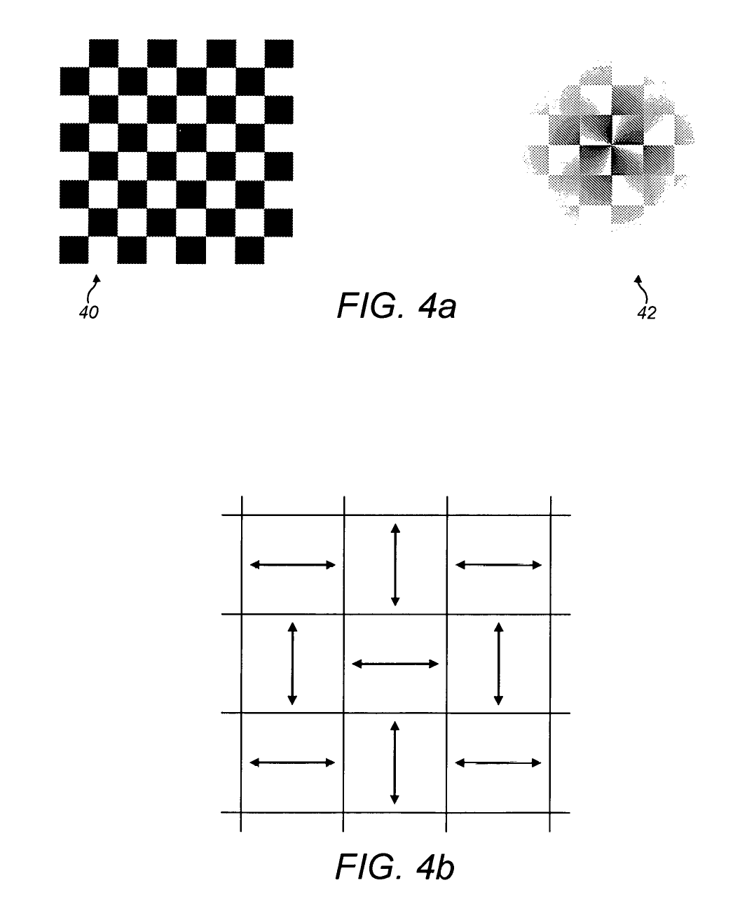

FIG. 4a shows an input pattern 40 which can be created using the apparatus of FIGS. 2 and 3. The input pattern 40 has the form of a checkerboard. The black and white squares of the checkerboard represent orthogonal polarization orientations and not luminance. The black areas may be horizontally polarized, while the white areas are vertically polarized. As mentioned above, the luminance of the display area should ideally be substantially uniform. FIG. 4b shows diagrammatically the individual liquid crystal cells for a portion of the checkerboard input pattern 40 and indicates their respective polarization orientation. FIG. 4a also shows a mathematical simulation 42 of the expected appearance to a healthy observer of the polarized checkerboard input image 40 when viewed at a predetermined distance. The predetermined distance may be chosen such that there are a number of boundaries between squares visible to the polarization sensitive region of the macula and depends upon the size of the input pattern. For example, an area of 3×3 squares or an area of 4×4 squares may occupy 3-5 degrees of visible angle. The exact visible angle at which the contrast in polarization states is clearest depends upon the individual viewing the input pattern.

The tendency of the retina to adapt so as to cause the perception of polarization to fade is overcome by involuntary saccadic movement of the eyes. The abrupt saccadic shift of eye fixation between adjacent areas of orthogonal polarization illuminates the macula alternately with orthogonally polarized light. The checkerboard pattern creates a more complex polarized image which is more distinctive than an isolated example of Haidinger's brush. This image may therefore be easier for some people to perceive. It may also act as a more sensitive test of macular function.

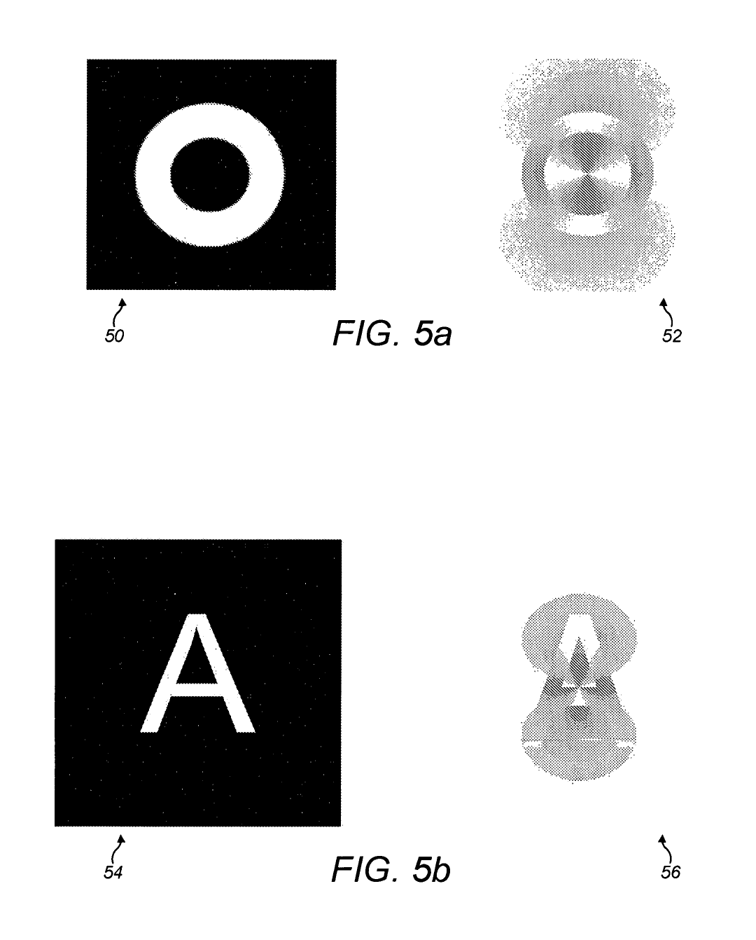

To aid in macular function diagnosis, more complex base images may be used representing generally recognizable patterns such as symbols, optotypes, alphanumeric or other characters. FIG. 5a shows an input pattern 50 which can be created using the apparatus of FIGS. 2 and 3. The input pattern 50 has the form of an annulus. As with the input pattern of FIG. 4a, the black areas may be horizontally polarized, while the white areas are vertically polarized. FIG. 5a also shows a mathematical simulation 52 of the expected appearance to a healthy observer of the polarized input image 50 when viewed at a predetermined distance. FIG. 5b shows a further example of an input pattern 54 in the form of the letter “A” and a mathematical simulation 56 of the expected appearance to a healthy observer. Using shapes or alphanumeric symbols is advantageous since these are easily recognized and described by most users. For example, the apparatus 2 may be used by an ophthalmologist or other health care professional as part of a more general sight or eye health test, and patients are accustomed to recognizing alphanumeric symbols in these circumstances. An observer can typically maintain a clear perception of the polarized image because of involuntary saccadic movement of the eyes. Such movement ensures that the polarization of light incident on a particular area of the macula is constantly changing.

In an active mode of operation, the apparatus 2 is controlled to produce different polarization patterns in succession or to alternate between two or more different polarization patterns. The principle of operation of the active mode is the same as for the static mode, however the active mode does not rely on the user shifting their gaze or on involuntary saccadic movement of the eyes to maintain a clear perception of a polarized image. Instead, the abrupt change in the generated polarized image may be used to negate the Troxler effect. The use of liquid crystal cells allows different polarized images to be presented to the retina with no apparent change in luminance. The images may also be made to alternate at any suitable frequency.

The simplest embodiment of the active mode of operation has a single area having uniform linear polarization. The orientation of the polarization is abruptly switched by 90 degrees at a desired frequency. In this case, an observer would see a single example of Haidinger's brush which switches between two orthogonal orientations. A device capable of implementing this embodiment can be created very cheaply since it requires only a single polarizing structure which can be switched between orthogonal states, therefore the LCC array and control circuitry can be greatly simplified.

In general, it is likely that more complex polarization patterns will be used, since this enables easier perception of the polarization patterns and can lead to a more sensitive assessment of macula function.

Referring to FIG. 6, a number of different polarization patterns and simulation of their respective expected appearances are shown. These patterns may be used in both passive and active modes and allows the health of the observer's macula to be investigated in greater detail. The pattern in active mode is more readily perceived than in passive mode therefore passive mode may be a more sensitive test of macular function. For example, alternating between the polarization patterns labeled 5b and 6b produces patterns alternating between those labeled 5s and 6s. The frequency of this switching may be varied to test the temporal response of the observer. As a further example, switching between the polarization patterns labeled 7b and 8b alternately produces the appearance of a pair of hourglasses (or this might be described as a “rounded cross”) and a thin “X”. The relative clarity of these two images may be used to infer the responsiveness of different areas of the macula.

The apparatus 2 can be embodied in a number of different ways as will now be described and may be used in static, active or combination mode in each of these ways.

In some embodiments, the apparatus 2 is a portable device. As the controlling circuitry and software is relatively simple, the device can be miniaturised and may for example be the size and thickness of a credit card or smaller. Such a device would be suitable for personal ownership and self testing. For example, the device may have a simple user interface comprising an on/off input and one or more other user inputs for cycling between patterns, for switching between a static mode and an active mode or for adjusting a pattern cycling frequency. The device may be preloaded with software capable of controlling the device to display a number of different polarization patterns and the user may cycle through these patterns using the inputs provided. The software on the device may be updatable, such that a user can download new patterns. The device may have an internal power source such as a replaceable battery or a rechargeable power cell. Thus a person who it at risk of developing a macula degenerative disorder can use the device to perform regular self testing, thus enabling the early detection of macula degeneration. For example, the user may test each eye daily. The test should take no more than 30 seconds to perform. Any change in the appearance/loss of perception of images would indicate the subject needs further examination.

In some other embodiments, the apparatus 2 is a larger screen, for example the size of a computer monitor or television. Such a screen may be used by an ophthalmologist or other health care professional when performing an eye health assessment. Subjects would view the screen at some distance, e.g. in the same way that a visual acuity chart is used. A subject is presented with a familiar pattern that is encoded through polarization. They are asked to identify the image. A positive response is indicative of normal macular function. A negative response such as “observing a blue light with no movement”, identifies that that subject requires a more detailed eye examination including assessment of the macula. It may also be possible to quantify a subject's retinal response. The contrast between different polarization states is maximised by viewing adjacent (in time or space) orthogonally polarised fields. The contrast and therefore ease of perception is lessened for reduced differences in polarization angle between the states shown to the viewer. Hence, by adjusting the polarization differences the visibility of the polarization contrast can be controlled from which perception limits can be established and quantified for each individual.

This size of screen could also potentially be placed in a public place to increase awareness of macula degenerative disorders and allow members of the public to test themselves.

The active mode of operation allows the temporal response of an observer to be quantified, by changing the rate of switching between patterns. For example a rate of ˜2 Hz is expected to create maximum perception between polarization states, whereas at a rate of ˜35 Hz, the limit of temporal resolution is approached, rendering the phenomenon practically invisible. In active mode of operation the polarized display is typically modulated at a frequency in the range 1-10 Hz with a 50% duty cycle.

In addition, the contrast can be reduced by reducing the level of polarization of the presented light. The introduction of non-polarized light or other polarized components of differing or random rotation states can be used to reduce the polarization contrast in a quantitative manner. For example, an optical retarder with variable orientation could be introduced. The optical retarder (or waveplate) may be tunable by an operator of the device in a quantifiable manner. This can advantageously lead to a quantifiable assessment of an observer's level of polarization perception. The optical retarder may be integral with the apparatus 2 shown in FIG. 2 and its orientation may be controlled manually or electronically via an input button, transducer or touch sensitive screen area integral with the apparatus.

A maximisation of polarization perception can be achieved by a combination of dynamic and spatial polarisation manipulation. Moving the polarization pattern either towards or away from the viewer, for a specific pattern, reduces the image visibility. This further enables eye diagnostics.

The system can be combined with a wavefront corrective or other device to enable observing the polarization as reflected from the retina directly using any of the previously described variants.

A further application is in the measurement of corneal birefringence. Switching the image between one form of elliptically polarised state and another creates a similar level of polarization contrast as described in the previous embodiments. However in this case the technique can also be used to measure the levels of birefringence within the cornea.

A further application is the stimulation of visually evoked cortical potentials (VECP) whereby a visual stimulus causes alteration in the electrical activity of the brain. This is a routine objective test of visual function using alternating luminance targets (e.g. luminance check patterns as in FIG. 4a). Light polarization-induced VECP have the advantage of being specifically generated at the level of the macula and thus test the visual system pertaining only to central vision.

A further application is the stimulation of electrical activity in the retina as detected for example by the electroretinogram or pattern electroretinogram. As with the VECP this has the advantage of being an objective test of the function of the macula of the retina.

Non medical applications may be the use of the phenomenon on small, medium or large-scale to enhance visual images for example in entertainment and other media applications.

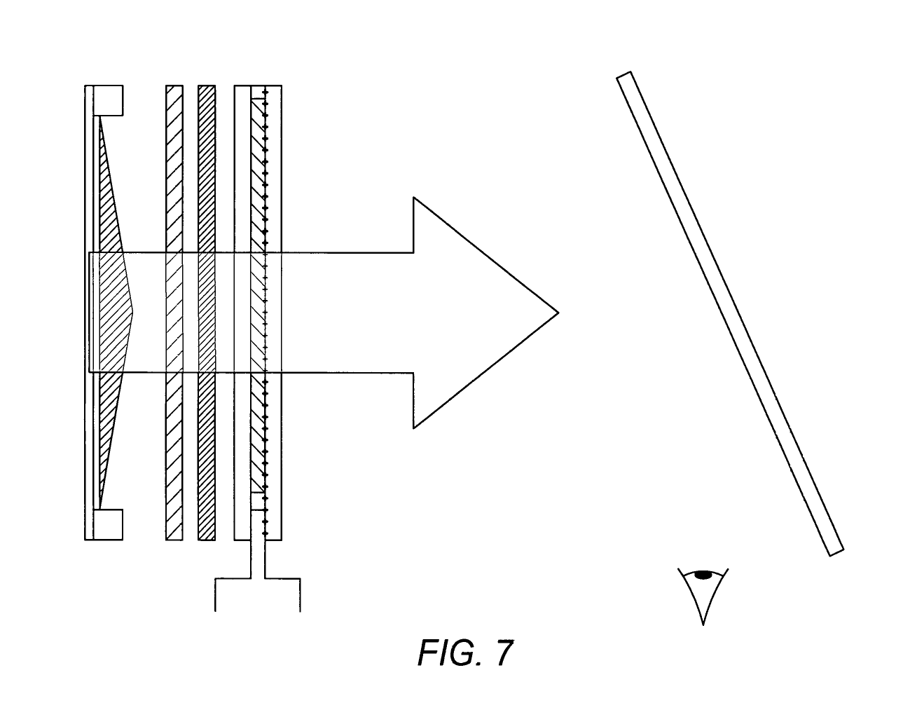

FIG. 7 illustrates a further embodiment in which the polarized images are projected onto a polarization preserving surface before being viewed by one or more observers. In FIG. 7 the screen and viewing angles are exaggerated and may be reduced to near-zero. This reflective mode enables a very large scale system to be constructed for example polarization patterns can be projected onto a cinema sized polarization preserving screen.

In a further embodiment of the static mode of operation, each light source e.g. each LED 4, can have an individual polarizing component to create the required polarization pattern, thus replacing the need for a LCC layer.

In a further embodiment of the invention, a polarization pattern may be embossed or otherwise encased within a suitable material without the need for any electronic components. For example, a flexible structure may be created comprising a polarizer, optionally a monochromator or other type of colour filter and a twisted nematic liquid crystal cell layer having a predefined polarized structure. This structure may be incorporated into a suitable card/material which can then be used to create a display of suitable size. For example, a user could hold the structure against a white LCD screen (which may also remove the need for a polarizer layer) or hold the structure up to a blue sky to see the polarization patterns. Alternatively, the structure may be placed in or in front of a projector.

In a further embodiment of the invention the subjects are not humans but animals that are known to be sensitive and/or responsive to polarized light. The polarization stimuli can be used to modify/ modulate behavior, for example as a means of capturing (e.g. insect traps, fishing). Furthermore such embodiments can be used in studying and investigating polarization vision in non-human organisms.

In a further embodiment of the invention, paired polarization patterns may be presented to each eye as in a stereogram. Each image is sufficiently disparate so as to produce a three-dimensional perception. This may require the observer to view the polarized image from a particular location or range of locations. A further stimulus pattern could be polarization patterns analogous to random-dot stereograms. A further stimulus pattern could be a single image but constructed and viewed as an auto-stereogram (single image random dot stereograms, SIRDS).

The above described device has many potential uses. These include:
Self testing of the ability to perceive the polarization of light
Fundamental (physiology) studies or adapted for use in specialist research/academia (e.g. AMD) clinics.
Quantitative determination of contrast or temporal perceptive thresholds.
Determining the relative levels of macular lutein (Lt) and hence predict those at risk of wAMD by quantifying polarization perception. Xanthophyll, the birefringent pigment underlying the mechanism of polarization perception, varies in concentration between individuals and is thought to correlate with the propensity to develop wAMD.
Determining the retardation of the cornea and other ocular media. This is both of scientific interest as well as having potential clinical applications.
Determination of macular function in the presence of ocular media opacities. Dense cataracts and other treatable opacities of the transparent components of the eye reduce vision and prevent an adequate examination of the macula. In such cases it is important to know if macular function is normal so that visual outcome of surgery can be predicted. Polarization perception, in the simple case of HP, is unaffected by such opacities unless extremely severe and could be used as a test of macular function in such cases.
Use in pleoptics. Pleoptics is the treatment for developmental amblyopia (lazy eye) especially in association with eccentric fixation (abnormal use of a non-macular part of the retina for visual fixation).
Use as a stimulus to generate visually evoked cortical brain potentials and electroretinograms as objective tests of macular function and neural pathways of vision.

It should be noted that there is a fundamental difference between perception of polarization (essentially Haidinger's phenomenon) and the polarization perception phenomenon (PPP), defined herein, which is the perception of the boundary of adjacent areas with different polarization states (e.g. axis of linear polarization, ellipticity/handedness for elliptic polarization states, degree of polarization).

1. Apparatus for inducing polarization perception in an observer, the apparatus comprising:
a light transmitting part configured to transmit light of substantially uniform luminance and colour;
a polarization manipulation layer comprising a polarizing structure having one or more regions of uniform state of polarization and arranged such that light transmitted through the light transmitting part passes through the layer; and
means for causing discrete changes in the state of polarization of the polarizing structure.

2. Apparatus according to claim 1, wherein the light transmitting part comprises a light emitting assembly configured to emit light of substantially uniform luminance and colour and wherein the polarization manipulation layer is arranged such that light emitted from the light emitting assembly passes through the layer.

3. Apparatus according to claim 2, wherein the light emitting assembly comprises one or more light sources and a chromatic filter.

4. Apparatus according to claim 2, wherein the light emitting assembly comprises a linear polarizer.

5. An apparatus according to claim 1, wherein the polarizing structure has a plurality of regions and wherein each region has a uniform state of polarization which differs from the state of each adjacent region.

6. An apparatus according to claim 1, wherein the polarizing structure defines a polarization pattern and wherein the means for causing discrete changes in the state of polarization of the polarizing structure comprise means for producing a series of at least two different polarization patterns.

7. An apparatus according to claim 6, wherein the polarizing structure defines a polarization pattern and wherein the means for causing discrete changes is configured to cause two different polarization patterns to be produced alternately.

8. An apparatus according to claim 7, wherein a frequency at which the different polarization patterns are produced is in the range of 1-10 Hz.

9. Apparatus according to claim 6, wherein at least one of the series of polarization patterns comprises an array of differently polarized regions.

10. Apparatus according to claim 6, wherein at least one of the series of polarization patterns comprises one or more symbols.

11. An apparatus according to claim 1, wherein the means for causing discrete changes in the state of polarization of the polarizing structure comprises a controller for controlling changes in the state of polarization of the polarizing structure.

12. An apparatus according to claim 1, wherein the means for causing discrete changes in the state of polarization of the polarizing structure comprises one or more user inputs.

13. Apparatus according to claim 1, wherein the apparatus is a portable display device.

14. Apparatus according to claim 1, wherein the polarization manipulation layer comprises an array of liquid crystal cells.

15. Apparatus for inducing polarization perception in an observer, the apparatus comprising:
a light transmitting part configured to transmit light of substantially uniform luminance and colour; and
a polarization manipulation layer comprising a polarizing structure having a plurality of regions and arranged such that light transmitted through the light transmitting part passes through the layer;
wherein each region of the polarizing structure has a uniform state of polarization which differs from the state of each adjacent region.

16. Apparatus according to claim 15, wherein the light transmitting part comprises a light emitting assembly configured to emit light of substantially uniform luminance and colour and wherein the polarization manipulation layer is arranged such that light emitted from the light emitting assembly passes through the layer.

17. Apparatus according to claim 15, wherein the light transmitting part comprises a colour filter configured to receive ambient light.

18. Apparatus according to claim 15, wherein the plurality of regions of the polarizing structure define a polarization pattern.

19. Apparatus according to claim 18, wherein the polarization pattern comprises an array of linearly polarized regions.

20. Apparatus according to claim 18, wherein the polarization pattern comprises a symbol, which is optionally an alphanumeric symbol.

21. (canceled)

22. Apparatus according to claim 15, wherein the apparatus further comprises an optical retarder with variable orientation.

23. (canceled)

24. (canceled)

25. Apparatus for inducing polarization perception in an observer, the apparatus comprising:
a light transmitting part configured to transmit light of substantially uniform luminance and colour; and
a polarization manipulation layer comprising a polarizing structure having one or more regions and arranged such that light transmitted through the light transmitting part passes through the layer;
wherein each region of the polarizing structure has a uniform state of polarization which differs from the state of each adjacent region and wherein the apparatus is configured to vary the state of polarization of the polarizing structure wherein the variances are temporal and/or spatial.
